(19) United States
(12) Patent Application Publication
(10) Pub. No.: US 2025/0009301 A1
(43) Pub. Date: Jan. 9, 2025

(54) MICRONEEDLE ELECTRODE PATCH DEVICE

(71) Applicants: COMMISSARIAT A L’ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d’Heres (FR)

(72) Inventors: Andrew GROSS, Gieres (FR); Bastien DARMAU, Saint-Martin-d’Heres (FR); Isabelle TEXIER-NOGUES, Grenoble Cedex 09 (FR)

(73) Assignees: COMMISSARIAT A L’ENERGIE ATOMIQUE ET AUX ENERGIES ALTERNATIVES, Paris (FR); CENTRE NATIONAL DE LA RECHERCHE SCIENTIFIQUE, Paris (FR); UNIVERSITE GRENOBLE ALPES, Saint-Martin-d’Heres (FR)

(21) Appl. No.: 18/764,556

(22) Filed: Jul. 5, 2024

(30) Foreign Application Priority Data
Jul. 6, 2023 (FR) 2307214

(51) Int. Cl. A61B 5/00; A61B 5/145; A61B 5/1473; A61L 31/04; A61L 31/14

(52) U.S. Cl. CPC A61B 5/685; A61B 5/14514; A61B 5/1473; A61L 31/041; A61L 31/145; A61B 2560/0214

(57) ABSTRACT

A device having a polymeric support with a surface for contact with skin, an array of solid polymeric microneedles integral with the support and projecting outward from the surface of the support provided for contact with the skin, and an electrode. The microneedles and the surface of contact of the support with the skin are formed of a crosslinked hydrogel, non-electron-conductive in the dry state and electrolyte-conductive upon contact with an aqueous fluid. The electrode is arranged in contact with the hydrogel provided to swell upon contact with an aqueous fluid and is free of direct contact with the skin. A method for preparing the device and uses thereof are also described.

### TECHNICAL FIELD

The present invention relates to the field of devices of use in particular for the characterization and/or electrochemical and transdermal stimulation of physical parameters (pH, conductivity, temperature) and/or of biomarkers in interstitial fluids. As is set out in detail below, these devices can also be considered for the conversion and/or storage of energy, techniques employing at least two electrodes in an electrochemical cell with an electrolyte between the electrodes, but also for the transdermal release of active agents in particular for therapeutic purposes, a technique of passing current between two electrodes through the skin or between subcutaneous tissues.

In particular, such a device is in the form of a microneedle (MN) patch coupled to at least one electrode.

### PRIOR ART

The advantage of devices for transdermal analyses, and more particularly of microneedle patches, is that once they are inserted into the skin, the one or more electrodes that they integrate can carry out a measurement of an analyte or a work such as the contraction of a muscle without necessary effort.

However, the methods of integration between microneedle patches and electrodes in order to obtain such devices are at present relatively complex.

A first type of integrated device combines various distinct microneedle patches, each patch being coupled to or constituting a different electrode like that described in [Ref. 1] Sanjiv Sharma et al. Anal Bioanal Chem, 2016, 408, 8427-8435. In another embodiment, several patches, each provided for the characterization of a specific biomarker, are combined in a single device [Ref. 2] Farshad Tehrani et al. Nature Biomedical Engineering, 2022. In these examples, the microneedles of each patch are treated on the external surface to constitute an electrode on each patch, with a different treatment for each patch in order to constitute different electrodes. During insertion of the needles into the skin, it is therefore not possible to avoid the risk of delamination and/or release of the materials, usually non-biocompatible metallic materials, constituting these electrodes. Moreover, the realization of devices combining several patches is complicated.

Devices have also been produced having a single patch but in which microneedles are still treated differently on the external surface in order to constitute different types of electrodes. Thus, the document [Ref. 4] US 2006/0264716 describes a microneedle patch made of Si/SiO (doped), of which some microneedles are coated with Ag/AgCl (“RE”) and others with multilayer coatings, comprising particles of Rh, Ag/cellulose acetate+glucose oxidase, and with a protective membrane layer of Nafion or PTFE to constitute the working electrode. The documents [Ref. 5] Hazhir Teymourian et al. Anal. Chem., 2020, 92, 2, 2291 2300 and [Ref. 6] A M Vinu Mohan et al. Biosensors and Bioelectronics, 91, 2017, 574-579l propose, according to another variant, placing these electrodes (working/transduction element, reference, counter-electrode) inside the internal channel of a microneedle. The microneedle withdraws the interstitial fluid by capillary action and brings it into contact with the electrode for the measurement. However, these two variants with a single patch also require a complex, multi-step manufacturing process and they do not make it possible to avoid the abovementioned risk of delamination either, since there remains a risk of the constituent material of the electrodes being transported into the inside of the analyzed tissue, via the interstitial fluid that is in direct contact with the electrodes.

Microneedle patches have also been proposed that further comprise one or more fluidic channels which are disposed behind the microneedles and into which the electrodes are inserted [Ref. 7] US 20090099427 and [Ref. 8] US 20140336487. Nevertheless, in this variant, there still remains the risk of toxicity in the event of release of the electrode material, which is not always biocompatible, into the interstitial fluid bathing the tissues. The production of a fluidic channel further complicates the manufacture of this type of device. Finally, [Ref. 3] Peyman G et al. ADV Health Mater. 2022 describes a device consisting of three polymeric microneedle patches, one of which (that of the working electrode) is based on a swelling and conductive hydrogel, in order to obtain a device for non-enzymatic measurement of glucose. Unlike [Ref. 1] and [Ref. 2], this time the working electrode is a microneedle patch made of crosslinked hyaluronic acid/dopamine hydrogel, supplemented with PEDOT:PSS (PEDOT: poly(3,4-ethylenedioxythiophene), a conductive polymer, PSS: poly(styrene sulfonate)) and nanoparticles of Pt and Ag (formed in situ by oxidation of Ag+ and Pt+ by dopamine) in order to increase the hydrogel conductivity. Due to the presence of PEDOT:PSS and of the Pt and Ag nanoparticles, the material is not only an ion conductor, but also an electrical conductor, and the MN patch constitutes an electrode in its own right.

Such a system therefore has the drawback of not lending itself to the integration of a counter-electrode (CE) or of a reference electrode (RE), otherwise it will be short-circuited.

Consequently, there remains a need for a device which is of particular use for transdermal electrochemical measurement, coupling at least one microneedle (MN) patch with one or more electrodes, and which is free of any risk of toxicity with respect to the subject on whom this measurement is performed.

There also remains the need for such a device which is compatible with the use of different combinations of electrodes, for example working electrodes (WE), reference electrodes (RE) and counter-electrodes (CE), and which, as such, is not itself an electrical conductor, as opposed in particular to a patch formed of a PEDOT:PSS conductive hydrogel as described in [Ref. 3].

There also remains a need for such a device to be of non-complex manufacture.

The present invention aims precisely to meet these needs.

### SUMMARY OF THE INVENTION

Thus, according to one of its aspects, the present invention relates to a device comprising at least one polymeric support 1 having a surface 4 provided for contact with skin 2, at least one array of solid polymeric microneedles 3 integral with said support 4 and projecting outward from said surface 4 of the support 1 provided for contact with said skin 2, and at least one electrode 9, characterized in that said microneedles 3 and at least the surface 4 of contact of said support 1 with said skin 2 are formed of a biocompatible crosslinked hydrogel, non-electron-conductive in the dry state and electrolyte-conductive upon contact with an aqueous fluid, and the, an or said electrode(s) 9 is (are) arranged in contact with the hydrogel provided to swell upon contact with an aqueous fluid and is (are) free of direct contact with the skin 2.

Unexpectedly, the inventors have thus observed that the coupling of an electrode or electrodes to a support comprising at least one array of microneedles can be performed under conditions allowing the abovementioned expectations to be met, provided that this substrate is in particular formed of a hydrogel according to the invention, namely capable of changing from a non-electron-conductive state when in a dry state to an electrolyte-conductive state when swollen by an aqueous fluid.

More precisely, and as will be seen from the examples below, the microneedles 3 of the device according to the invention advantageously possess, in the dry state, the mechanical strength necessary for perforating a body tissue, in particular to depths of between 200 and 2000 μm in the case of human skin, in order to access its interstitial fluid (ISF) efficiently. When the crosslinked and dry hydrogel constituting these microneedles 3 is in contact with this ISF, it swells, and the fluid to be analyzed will diffuse there by osmotic pressure difference and by capillarity. Thus, from an electrical point of view, the microneedles 3 and the part of the support 1 integral with the microneedles 3 can be considered, when they are impregnated with ISF, as becoming an extension of the tissues with the ISF and then perform an electrolyte function. The electrode or electrodes 9 placed in contact with this hydrogel swollen with ISF can then carry out a measurement or operation faithful to that which would be carried out if they were inserted directly into the skin 2, but advantageously in a minimally invasive manner.

According to a variant embodiment, the, an or said electrode(s) 9 is (are) integrated into the crosslinked hydrogel constituting the support 1, which is in direct contact with the rear of the microneedles 3.

According to another variant embodiment, the, an or said electrode(s) 9 is (are) integrated into the crosslinked hydrogel constituting the microneedle(s) 3. In particular, they are arranged there so as to have contact only with the ISF, which diffuses there by capillarity and/or by difference in osmotic pressure. Thus, as opposed to other devices of the prior art, the thus arranged electrodes 9 of the device of the invention do not have direct contact with the skin 2 on which the analysis is performed.

According to yet another variant embodiment, the device according to the invention integrates at least one electrode in the constituent crosslinked hydrogel of the support 1 in direct contact with the rear of the microneedles 3, and at least one electrode in the constituent crosslinked hydrogel of the microneedles 3.

The device according to the invention is therefore advantageous in several respects.

Its constituent crosslinked hydrogel is compatible with the integration of any electrode (porous or non-porous) for the detection of analytes, ions and biomarkers, especially a bioelectrode with an enzymatic coating on the electrode. At the surface of and optionally deep within the electrode(s), this hydrogel thus provides an aqueous environment suitable for operating under optimal conditions and for prolonging the lifetime/stability of the electrode(s) and thus the lifetime/stability of the transdermal device.

Only its crosslinked hydrogel component is in direct contact with the biological tissue being analyzed, and therefore the risk of delamination of the coating and/or constituent material of he electrode (e.g. mediator, enzyme, AgCl layer, etc.) upon insertion of the microneedles into the tissue is avoided.

The risk of detachment of a component or components of an electrode is also avoided on account of the latter being integrated within the crosslinked hydrogel of the device according to the invention. Similarly, the voltage drop (or ohmic drop) caused by the resistance of the solution between working and reference electrodes can be minimized by intimate integration within the same microneedle patch.

The positioning of the electrode close to the rear of the tips of the microneedles and/or in microneedles themselves also limits the distance that the analyte, ion, proton, biomarker must travel and/or any mass transport limitation that could affect the performance of the sensor, including reduced latency time.

According to a preferred variant, the assembly composed of support 1 and microneedles 3 is formed of said biocompatible crosslinked hydrogel which is not electron-conductive in the dry state and which swells upon contact with an aqueous fluid.

In particular, the crosslinked hydrogel is free of any metallic constituent and electron-conductive polymer.

In particular, the device according to the invention is in the form of a microneedle (MN) patch.

According to another of its aspects, the present invention relates to a method for preparing a device according to the invention, in particular by micro-molding, and comprising at least the crosslinking of at least one polymer, preferably a biopolymer, in order to form said crosslinked hydrogel, characterized in that the one or more electrodes 9 of said device are integrated by being placed in contact with said polymer prior to or simultaneously with its crosslinking.

In particular, the one or more electrodes 9 are placed in contact with the aqueous formulation containing at least said non-crosslinked polymer and are subjected there to mechanical pressure in order to position them at depth in proximity to the base of the microneedles 3 and/or at depth in the microneedles 3, prior to or simultaneously with the crosslinking.

According to yet another of its aspects, the present invention relates to the use of a device according to the invention for energy conversion, for energy storage or for transdermal electrochemical measurements, in particular for characterizing at least one analyte in an interstitial fluid 6.

Thus, the invention relates more particularly to a method for detecting and/or assaying at least one analyte in an interstitial fluid 6 of a patient, comprising at least placing the microneedles 3 of a device according to the invention in contact with an interstitial fluid 6 of said patient under conditions that are conducive to the swelling of said constituent crosslinked hydrogel of the microneedles 3 by this fluid 6 and to the diffusion of this fluid 6, by a difference in osmotic pressure and/or capillarity, as far as the electrode(s) 9 of said device.

Other features, variants and advantages of the subjects of the invention will emerge more clearly on reading the description, the examples and figures which follow, which are given by way of illustration and do not limit the invention.

In the remainder of the text, the expressions “of between . . . and . . . ”, “ranging from . . . to . . . ” and “varying from . . . to . . . ” are equivalent and are intended to mean that the limits are included, unless otherwise stated.

### BRIEF DESCRIPTION OF THE DRAWINGS

FIG. 1 shows a schematic example of a device according to the invention.

FIG. 2a shows the chronoamperogram of a working electrode (WE) integrated within a Dex-MA MN patch (example 2), showing the changes in the current after successive additions of glucose at regular times at −0.1 V versus pseudo-reference Ag/AgCl.

FIG. 2b shows the calibration curve of the same working electrode (WE) as in the preceding figure, integrated within a Dex-MA MN patch (example 2), integrated within the same MN patch.

FIG. 3 shows the results obtained at the end of the cytotoxicity tests detailed in example 5.

### DETAILED DESCRIPTION

As will be clear from the above, the invention aims to propose minimally invasive devices that can intervene transdermally, either continuously or non-continuously, in particular for the characterization and/or stimulation of physical parameters (pH, conductivity, temperature) and/or the transdermal detection of biomarkers in interstitial fluids.

More particularly, this device comprises at least one polymeric support 1, at least one electrode 9, and microneedles 3 arranged on one of the faces of the support. The microneedles 3 penetrate into a biological tissue, as far as the interstitial fluid 6 thereof, in which it is precisely sought to electrochemically characterize at least one physical parameter and/or one chemical or biological species or to effect electrical stimulation, for example.

In the device according to the invention, the microneedles 3 are solid. In other words, they are not hollow but are formed of crosslinked hydrogel in accordance with the invention and, where appropriate, of an electrode 9 integrated into this hydrogel.

The microneedles 3 preferably have a length varying from 200 to 3000 μm.

In a variant embodiment, the device according to the invention can comprise several arrays of microneedles 3 and/or several electrodes 9.

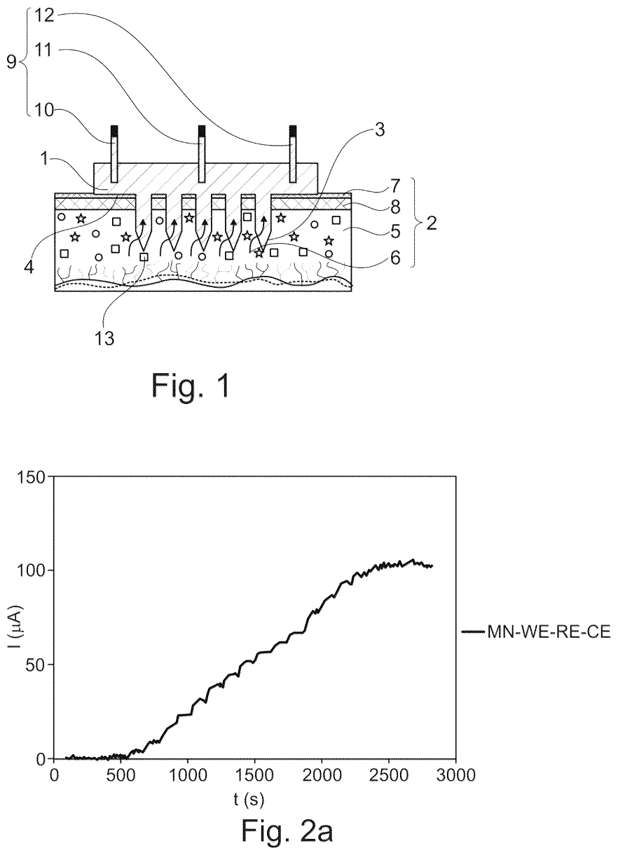

In particular, the device according to the invention consists of a microneedle (MN) patch, in particular as illustrated in FIG. 1.

The device in FIG. 1 is formed by a body made of a crosslinked hydrogel which, before use, is in a dry state. The body comprises a support 1, and microneedles 3 which project from a surface 4 of the support 1 in contact with the skin 2 of a patient and which have penetrated into the dermis 5 of the skin of the patient, through the stratum corneum 7 and the epidermis 8.

According to the device of FIG. 1, the device comprises a reference electrode 10 for defining a reference electric potential, a working electrode 11, and a counter-electrode 12 which defines an electrical circulation circuit with the working electrode 11, so as to maintain a constant voltage in the reference electrode 10.

The reference electrode 10, the working electrode 11 and the counter-electrode 12 are integrated in the volume of the support 1.

The interstitial fluid 6 present in the dermis 5 comprising the biomarkers 13 diffuses toward the electrodes 10, 11, 12 through the microneedles 3 and then into the support 1, forming a crosslinked hydrogel filled with electrolyte (interstitial fluid).

The electrical connection, not shown in FIG. 1, can be formed by a wire made of an electrically conductive material (for example a silver-plated copper wire) adhering to the back of the electrode in question with a conductive paste (such as carbon paste).

### Crosslinked Hydrogel

Within the meaning of the invention, a crosslinked hydrogel is a hydrophilic and insoluble three-dimensional polymer. Its crosslinking is obtained by physical and/or chemical bonds, preferably chemical bonds, existing between polymer chains.

In the dry state, the crosslinked hydrogel possesses the hardness necessary for the penetration of the microneedles 3 of which it is composed, but on the other hand it is not capable of ensuring the circulation of electrons itself. In other words, it is not an electron conductor or semiconductor. It is thus free of any metallic constituent and organic conductive polymer such as PEDOT, poly (pyrrole) or poly (aniline).

Moreover, this dry crosslinked hydrogel is capable of swelling upon contact with a fluid, especially an aqueous fluid such as water or a biological fluid. It can therefore absorb and retain a large amount of a fluid. This hydrogel swelling rate (SR) is generally defined by the ratio between the weight of the swollen hydrogel and the weight of this same hydrogel in the corresponding dry state. More precisely, the fluid diffuses inside the crosslinked hydrogel by capillarity phenomena and/or osmotic pressure difference between the fluid and the hydrogel. Thus impregnated, the crosslinked hydrogel makes it possible to bring the aqueous fluid to be characterized into contact with, in particular, the working electrode which it integrates. In other words, the swelling of the hydrogel by the interstitial fluid 6 causes the device according to the invention, insulating in the dry state, to become an electrolytic matrix (ionic conductor) and makes it possible to form an electrolytic bridge between the tissue in which the microneedles 3 are inserted and the electrode or electrodes 9 that it contains. In contrast to devices of the prior art, the crosslinked and swollen hydrogel according to the invention also acts as a filter isolating the one or more electrodes 9, which it integrates, from any possible contact with the biological tissue. The structural network of the crosslinked gel is such that, in the swollen state, it makes it almost impossible for any small fragments of electrode to migrate toward this tissue. Any risk of toxicity due to the electrodes 9 is therefore excluded or greatly minimized.

The crosslinked hydrogel according to the invention is also biocompatible.

Within the meaning of the invention, a biocompatible hydrogel is a hydrogel which, by its chemical nature, is not such as to cause an adverse effect, such as inflammation, in the biological tissue that is analyzed.

The biocompatible aspect of the hydrogel is an asset, because only it is in direct contact with tissues as a component of the microneedles 3 penetrating the skin 2 of a subject. Thus, the hydrogel constituting the microneedles 3 and the support 1 acts as a screen between the skin 2 and the one or more electrodes 9 associated with it in the device according to the invention. Moreover, if the hydrogel, although crosslinked, remains bioresorbable over the long term, the polymer microneedles 3 will not pose any risk if they break in the skin 2 during use of the device, since the component from which they are made will be able to be resorbed over time without damaging the tissues.

Finally, in view of its biocompatibility, the hydrogel can play a beneficial role as regards the stability of bioelectrode(s) 9 likely to be associated with it in the device according to the invention. It constitutes a biocompatible environment for the enzymes, can reduce their denaturation and can allow favorable mass transport.

As has already been specified, the crosslinked hydrogel according to the invention advantageously possesses good swelling properties (swelling in 24 hours greater than 10%, preferably greater than 20%, more preferably more than 50%) and, in the dry state, good mechanical hardness for piercing the skin (Young's modulus greater than 10 kPa).

The crosslinked hydrogel according to the invention can be obtained by crosslinking any type of polymer which, once crosslinked, forms, in contact with an aqueous fluid, an insoluble and infusible hydrogel.

In particular, the crosslinked hydrogel according to the invention can be obtained by crosslinking one or more synthetic polymers, one or more biopolymers, where appropriate chemically modified to be crosslinkable, or a mixture thereof.

Within the meaning of the invention, the term “biopolymer” denotes a polymer derived from renewable natural resources (a material also referred to as “natural”). The biopolymers considered according to the invention have the advantage of possessing, in addition to very good compatibility with human skin, biodegradability as such.

This term biopolymer thus covers natural polymers such as, for example, a protein or a polysaccharide, but also derivatives of natural polymers such as, for example, those derived from a chemical modification of these natural polymers to make them crosslinkable or compatible with a specific crosslinking mode.

This chemical modification usually consists of introducing, into the structure of the polymers to be modified, crosslinkable units chosen in particular from thiol, divinylsulphonyl, maleimide, azide, alkynyl, alkenyl, acrylate, methacrylate, aldehyde, norbornenyl, oxy-amine and mixtures thereof, and in particular from acrylates, methacrylates and mixtures thereof.

By way of illustration of synthetic polymers suitable for forming a crosslinked hydrogel suitable for the invention, mention may be made in particular of crude poly(ethylene glycols) (PEG) or those modified by different chemical functions, for example poly(ethylene glycol) diacrylic (PEGDA), poly(ethylene glycol) dimethacrylic (PEGDMA), poly (vinyl pyrrolidone) (PVP), poly(methyl vinyl ether maleic acid)-PEG, PVA-MA, -PLA, -PLLA, -PGA, -PLGA and their derivatives.

By way of illustration of biopolymers suitable for forming a crosslinked hydrogel in accordance with the invention, mention may be made in particular of polyhydroxy acids, for example polyglycolic acid (PGA) and polylactic acid (PLA), also known as polylactide; polysaccharides, for example cellulose, chitin and starch, and proteins or peptides, for example collagen, gelatin, elastins, fibroins, maize zein, the silk of various species of silkworms, keratin and their derivatives.

According to a preferred variant embodiment, the crosslinked and biocompatible hydrogel considered according to the invention is obtained by crosslinking at least one biopolymer chosen from polymers such as chitosan, dextran, alginates, collagen, gelatin, agarose, chitin, polyhydroxyalkanoates, pullan, starch, amylose, amylopectin, cellulosic, in particular carboxymethylcellulose, and hydroxypropylcellulose, hyaluronic acid, gellan gum, xanthan gum, for example the derivatives of these polymers resulting from their chemical modification to make them crosslinkable or compatible for a specific crosslinking mode, and combinations thereof.

According to a particular embodiment, this biopolymer is or derives from at least one polymer chosen from alginates, hyaluronic acid, carboxymethylcellulose, chitosan, dextran and derivatives thereof, and preferably dextran.

In particular, the hydrogel is a crosslinked dextran hydrogel.

Within the meaning of the invention, the term dextran covers dextrans which have or have not been chemically modified in order to be crosslinked.

Thus, it may be advantageous to crosslink a dextran polymer chemically modified by acrylate and/or methacrylate units which are well known to be photopolymerizable.

These functional groups, which permit crosslinking by irradiation, are introduced with a given degree of substitution (DS). Conventionally, the DS corresponds to the number of polymerizable functional groups introduced on 100 monomer units. This DS can be determined by ¹H NMR. In the following, it is expressed as a percentage.

Thus, according to a particular embodiment, said hydrogel derives from the crosslinking of at least one dextran polymer modified by units chosen from acrylate, methacrylate, alkenyl and alkynyl, preferably a dextran polymer modified by methacrylate units, DexMA, and of DS varying from 5 to 65%.

### Electrodes and Their Mode of Integration

Another specificity of the device of the invention is based on the mode of integration of the electrodes.

As has been specified above, the electrode or electrodes 9 are arranged so that they do not have direct contact with the tissue of which the ISF 6 is being analyzed.

In general, they are fully integrated into the crosslinked hydrogel to be swollen by this ISF 6, with the exception of their end provided for the external electrical connection. Generally, this connection is located on the upper face or one of the lateral faces of the device.

Within the scope of the present invention, and in contrast to conventional devices, the one or more electrodes 9 are also not in communication with a channel to which the microneedles 3 would be connected.

As has already been specified above, they are instead disposed either close to and/or within solid microneedles 3 of the device.

According to a particular embodiment, they are integrated within the thickness of the support 1 located to the rear of the microneedles.

This mode of integration is illustrated in particular in FIG. 1.

As has been specified above, the device is compatible with the use of several electrodes 9 insofar as the crosslinked hydrogel constituting the device is not an electron conductor. These electrodes 9 can be chosen from reference electrodes, counter-electrodes, working electrodes such as ion selective electrodes (ISE), pH electrodes, electrocatalytic electrodes, bioelectrodes, micro-or nano-structured electrodes.

Thus, the device can comprise one or more electrodes 9 chosen from electrodes or bioelectrodes based on Pt, Au, Ag, Pd, Ni, Ir, graphitic carbon, amorphous carbon, graphene, graphene oxide, diamond, boron-doped diamond, nanotubes, doped semiconductor fibers, for example and preferably doped silicon, metal oxides, for example and preferably indium tin oxide, and electrodes made of conductive polymers, especially and preferably PEDOT and conductive fibers.

In fact, any type of electrode and/or electrochemical sensor in general (sensor, biosensor, photoelectrochemical sensor with electrode transparent in the visible range) can be integrated into a device according to the invention when the sensor in question requires diffusion of an aqueous electrolytic medium to the sensor.

The electrode 9 can be a macro, micro or nanoelectrode.

The general shape of the electrode 9 may or may not be plane. The electrodes 9 can comprise a sheet of carbon paper or of tissue or a sheet or a pad or a printed electrode, for example a screen-printed electrode. The electrodes of non-planar shapes comprise a Pt wire or a carbon fiber wire or a wound electrode.

For example, these electrodes 9 can be chosen from electrodes or bioelectrodes based on Pt, Au, Ag, Pd, Ni, Ir, graphitic carbon, amorphous carbon, graphene, graphene oxide, diamond, boron-doped diamond, nanotubes, doped semiconductor fibers, e.g. doped silicon, metal oxides e.g. indium tin oxide, conductive polymer electrodes, e.g. PEDOT, and conductive fibers.

According to a first variant, the device comprises a single electrode 9 and in particular a working electrode. The term “working electrode” here designates an electrode to which a candidate compound (analyte, biomolecule, active agent) is electro-oxidized or electro-reduced with or without the intervention of a redox mediator. The term “redox mediator” designates an electron transfer agent that transfers electrons between a compound and an electrode directly or indirectly. A redox mediator is often incorporated in WE of the bioelectrode type (enzyme electrode; enzymatic electrode) to facilitate electron transfer between an enzyme (oxidoreductase) and an electrode in order to allow oxidation or reduction of a candidate compound. The WE of the (bio) electrode type can in particular play the role of (bio) electrochemical sensor. Such sensors are generally coupled in solution with a counter-electrode and a reference electrode. The sensor functions in such a way that a current is generated between the working electrode and the counter-electrode. Such electrodes are particularly advantageous for detecting and/or assaying a chemical or biological analyte, for example proteins, amino acids, viruses, hormones, medicaments, drugs, and in particular an analyte chosen from glucose, lactate, alcohol, nitrate, alanine, cysteine, pyruvate, glycerol, Ca2+, Mg2+, K+ ions, phosphate, urea, cholesterol, glutamate, hydrogen peroxide, hydrogen, etc. The working electrode is often metal or carbon based and is used for electro-oxidation or electro-reduction of the candidate compound and often with one or more catalytic or electrocatalytic elements such as noble metals, nanoparticles, organometallic molecules, and enzymes. They can in particular be working electrodes with recognition elements (often called ‘receptors’) to facilitate specific and selective detection and often with a polymeric element to facilitate properties such as mechanical properties, recognition and/or signal transmission.

It is also conceivable to combine several working electrodes respectively provided to characterize different chemical or biological analytes and/or physical parameters.

According to a second variant, the device can comprise a counter-electrode (CE). Such electrodes are particularly advantageous for establishing a circuit with a working electrode to which the current is applied or on which the current is measured. Electrodes of Pt, Ag, AgCl or other metals (Ti, Au, Pd, tin), metal oxides (IrO₂), carbon electrodes (vitreous carbon), conductive polymer electrodes (PEDOT), are auxiliary electrodes typically used in electrochemistry.

According to another variant, the device can comprise at least one reference electrode (RE). Such electrodes are particularly advantageous for measuring or monitoring the potential of an indicator or of the working electrode. Saturated calomel electrodes or silver chloride electrodes are reference electrodes typically used in electrochemistry.

According to yet another variant, the device comprises at least two or even at least three or even four electrodes 9. A device according to the invention can thus integrate, for example, one to five working electrodes for the detection of one to five different analytes with, where appropriate, a reference electrode and a counter-electrode (or hybrid RE/CE electrode). They may in particular be (bio)chemical sensors: potentiometric; amperometric; coulometric; impedimetric and/or bipotentiostatic.

A device according to the invention can also incorporate several sensors chosen from physical sensors (e.g. conductivity of the solution) and actuators for electrostimulation and administration of medicaments (electrophoresis, electro-osmosis), electrosynthesis, electroregeneration,

A device according to the invention can thus integrate two electrodes 9 of opposite charge for a physical measurement or activation (e.g. a current discharge).

A device according to the invention can also integrate two electrocatalytic electrodes of opposite charge (e.g. anode and cathode) 9 for the production or storage of energy. Of course, a device according to the invention and provided for a physical measurement or activation can also incorporate a single electrode 9.

The device according to the invention can also comprise an electrochemical detection device attached to said electrode or electrodes 9 via electrical connections, preferably surrounded by insulation. This detection device thus makes it possible to carry out measurements by potentiometry, cyclic voltammetry (CV), fast-scan cyclic voltammetry (FSCV), square wave voltammetry (SWV), pulsed voltammetry or chronoamperometry.

### Method for Preparing a Device According to the Invention

As has been specified above, the invention also relates to a method for preparing a device according to the invention, comprising at least the crosslinking of at least one polymer, in particular a biopolymer, in order to form said crosslinked hydrogel, characterized in that the electrode(s) 9 of said device are integrated by being placed in contact with said polymer prior to or simultaneously with its crosslinking.

In particular, during the contacting of the electrode or electrodes 9, the polymer is used in the form of a liquid or semi-liquid aqueous formulation, that is to say of viscous appearance, preferably having a viscosity varying from 1 to 100 Pa·s⁻¹ at 1 Hz when the applied shear stress is 0.2%. The polymer solution is preferably prepared in aqueous solution, with or without salts, such as NaCl, KCl, LiCl, or with or without buffer, such as a phosphate buffer. This liquid formulation, preferably aqueous, contains said polymer and preferably at least one crosslinking agent.

The electrode(s) 9 to be integrated are placed on the surface of this aqueous formulation containing at least said non-crosslinked polymer and are there subjected to mechanical pressure in order to position them at depth and close to the base of the microneedles 3 and/or within the constituent hydrogel of the microneedles 3, prior to or simultaneously with the crosslinking. In fact, at this viscosity, the one or more electrodes 9 with their contact can be easily inserted with a moderate force (slight pressure of the thumb, for example) in a precise and/or reproducible manner. They can be inserted such that the contact pick-up wires (or other electrical contacts) preferably exit the device from the top and/or the side of the support 1 integral with the microneedles 3.

After the insertion of the electrode(s) 9, the crosslinking is carried out or continued until a dry crosslinked hydrogel is obtained. To do this, the crosslinking operation is generally followed by a drying operation.

The crosslinking can be a chemical crosslinking. In the latter case, a crosslinking agent is also present in the liquid formulation containing the polymer(s) and preferably the biopolymer(s) to be crosslinked. This often slow method of crosslinking is conducive to the integration of the electrode(s) 9 during the crosslinking.

The crosslinking can also be triggered by a physical stimulus such as heat, gas exposure or irradiation (for example light, UV radiation, X-rays, gamma rays, microwave radiation).

According to a particular embodiment, the crosslinking is carried out photochemically.

Advantageously, the devices according to the invention can be prepared by a micro-molding technique using a silicone mold, itself obtained from a master model produced by micro-machining.

The liquid formulation, preferably aqueous, containing the biopolymer and preferably at least one crosslinking agent is poured into the mold obtained from this master model. After evaporation of the water or solvent from the solution, and crosslinking, this mold makes it possible to obtain the dry crosslinked hydrogel device, which can be removed from the mold.

### Use

According to a first variant, a device according to the invention can be used for electrical stimulation. Transdermal electrical stimulation is a technique that often involves sending an electrical pulse through the skin in order to perform work such as contraction of a muscle without necessary effort. This technique can be used in particular to treat diseases such as chronic pain and is widely used in the medical field. In particular cases, it makes it possible to facilitate the passage of molecules through the dermis for applications of delivery of species such as active principles, vaccines, etc. This technique consists in passing a current through the skin between two electrodes. Alternatively, electrochemical stimulation can be performed entirely by passing a current between two electrodes in the subcutaneous tissues. The use of a transdermal device also makes it possible to stimulate deeper layers of the skin than with conventional systems.

According to a second variant, a device according to the invention can be used for generating energy. It can be used, for example, as an enzymatic biofuel cell (or simply biocell). This type of cell uses enzymes to produce electrical power in complex media, for example, in interstitial fluid (ISF). The principle of the transdermal biocell is based on the integration, within the subdermal fluid, of two electrodes ensuring the operation of the generator: an electrode called an “anode” with one or more enzymes for catalyzing the electro-oxidation of the fuel(s) (e.g. glucose), and an electrode called a “cathode” for the electro-reduction of the oxidant, such as oxygen. The two electrodes are spatially separated in the same fluid. The cathode in a biocell often comprises an enzyme as a catalyst for this electro-reduction reaction. A particular and especially practical property for the application in an in vivo solution as an enzymatic biocell is that the selectivity of the enzymes immobilized on the electrode allows an electrochemical cell design without the need for a specific separating membrane (for example a proton exchange membrane). A biocell further comprises means for electrically connecting said biocell to an electrical receiver. In general, a transdermal biocell can provide a stand-alone energy solution for powering devices with a point-in-time electrical energy requirement, for example electronic devices such as sensors or stimulators.

According to another variant, a device according to the invention can be used for storing energy. It can, for example, be used as a supercapacitor (for example an electrochemical double layer capacitor) or as a hybrid enzymatic biofuel cell/supercapacitor device. This type of capacitor comprises two electrodes, often made of carbon, for example activated carbon or carbon nanotubes. These electrodes are impregnated with an electrolyte and spatially separated, for example by an insulating and porous membrane, so as to permit ionic conduction. A space charge region (called an electrical double layer) is formed at each electrode-electrolyte interface. In general, a supercapacitor can store energy and quickly provide the latter in order to power various electronic devices.

In another variant, the supercapacitor can be recharged by conversion to electricity by a (bio)cell without having to be connected to an external electrical power source.

A device according to the invention is advantageously of use for transdermal electrochemical measurements, in particular for characterizing at least one analyte in an interstitial fluid 6.

Within the meaning of the invention, the term analyte covers any chemical or biological species such as a medicament, a bioactive agent, a metabolite, a biomarker or an endogenous biochemical product.

Examples include, but are not limited to, glucose, sodium, potassium, alcohol, lactate (important for athletes), cortisol, urea, drugs (e.g. cannabinoids, amphetamines, cocaine and opioids) and nicotine metabolites (e.g. cotinine) and also the medicinal substances that a patient may be taking for one or more medical conditions.

Thus, the present invention also relates to a method for detecting and/or assaying at least one analyte 13 in an interstitial fluid 6, in particular in a non-invasive manner, comprising at least placing the microneedles 3 of a device according to the invention in contact with said interstitial fluid 6 under conditions that are conducive to the swelling of said constituent hydrogel of the microneedles 3 by said fluid and to the diffusion of this fluid, by a difference in osmotic pressure and/or capillarity, as far as the electrode or one of the electrodes 9 of said device.

The examples and figures that follow are provided by way of non-limiting illustration of the

### field of the invention

### Materials

Dextran T70 (Dex T70, MW=70,000 g/mol) and dextran T20 (Dex T20, MW=20,000 g/mol) from Pharmacosmos

Pigskin gelatin (gel strength 300, type A)

Bovine serum albumin (BSA, >95%) from Sigma-Aldrich,

The artificial interstitial fluid (ISF) was prepared by dissolving 22 g·l⁻¹ of BSA in a PBS buffer (PBS 1×: 137 mm NaCl, 2.7 mm KCl, 1 mm Na₂HPO₄, 1.8 mm KH₂PO₄, pH 7.4) and adjusting the pH to 7.4 if necessary.

The glucose solutions in PB (0.1 M Na₂HPO₄; pH=7.4) and the PBS were prepared at 1M by solubilizing the appropriate amount of glucose in PB or PBS and adjusting the pH to 7.4 if necessary.

### Example 1

### Preparation of Chemically Modified Dextrans

Methacrylated dextrans were synthesized according to the method described in the application FR 2,114,492.

The detailed protocol is as follows: Dextran (5 g) was dissolved in 100 ml of distilled water (DW) in a beaker. After complete dissolution, various equivalents of methacrylic anhydride of 0.0625 to 0.5 equivalent relative to the hydroxyl functions of the dextran were added dropwise to the polymer solution in order to obtain the desired degree of substitution (DS) of the polymer, i.e. 9, 18, 37 or 62%. The pH was then adjusted with NaOH (3 mol.l⁻¹) in order to be around 9-11 throughout the reaction. The solution was stirred at ambient temperature for 1 hour. Finally, the modified dextran was dialyzed against distilled water for 1 week (12-14 kDa membrane) and lyophilized. The white solid was stored at −20° C. before use.

### Characterization of Photocrosslinked Dextran-Methacylate Hydrogels

A PBS solution containing 20% (w/w) of Dex-MA, with a DS of 9, 18, 37 or 62%, and 1% (w/w) of (lithium phenyl-2,4,6-trimethylbenzoylphosphinate) LAP as photoinitiator was prepared in a 10 ml flask. 100 μl of solution were poured into a cylindrical Teflon mold 6 mm in diameter (without microneedle cavity), and the solution was allowed to air dry for 24 hours. The polymer was crosslinked by a first irradiation of the back of the patch at 405 nm for 1 minute (P=75 mW/cm2), followed by demolding of the dry polymer cylinder and a last irradiation at 405 nm for 1 minute on the other face of the cylinder.

These 4 materials were characterized by a swelling test.

To do this, the 4 dried photocrosslinked Dex-MA materials were weighed (weight W0). The samples were placed in a flask containing PBS (1×) at ambient temperature (21-24° C.). They were withdrawn every hour, and their surface was quickly wiped off with a paper towel in order to remove excess water. Thereafter, the samples were immediately weighed (Wt) and replaced in PBS for the following measurement points. This operation was repeated until no further change in weight was detected. The swelling rate (SR) was calculated according to SR= (Wt−W0)/W0. Three experiments were carried out in parallel for each material.

Table 1 below shows the mean values of the swelling rates thus determined.

TABLE 1

| Sample | |
|---|---|
| Dex-MA | Swelling rate (%) |
| DS = 62% | 43.9 ± 2.2 |
| DS = 37% | 55.2 ± 2.0 |
| DS = 18% | 84.2 ± 1.5 |
| DS = 9% | 98.0 ± 2.5 |

The maximum swelling rate (SR) for each formulation was reached in about 1-2 hours. An increase in the SR is correlated with a decrease in the DS.

The hardness of each of these 4 materials was also characterized by a compression test (in the dry state before swelling).

These compression tests were carried out using a TAXT.Plus texturometer (Stable Micro Systems, UK).

The compression rate was set at 0.5 mm·s⁻¹ with a maximum compression force of up to 50 N. The compression modulus values, E, were calculated as the slope of the curve of applied force versus displacement. Table 2 shows these values.

TABLE 2

| | Compression modulus |
|---|---|
| Sample | E (MPa) |
| Dex-MA DS = 62% | 159 ± 7 |
| Dex-MA DS = 37% | 167 ± 10 |
| Dex-MA DS = 18% | 161 ± 9 |
| Dex-MA DS = 9% | 155 ± 7 |

No alteration of the mechanical properties of the materials is observed. It is advantageous to note that the compression moduli of the photocrosslinked dried Dex-MA materials are well above 10 kPa, the threshold value for piercing the skin.

### Example 2

### Preparation of a Microneedle (MN) Electrode Patch

a) Preparation of a working electrode (WE) formed of carbon nanotubes and coated with electrochemical mediator (1,10-phenanthroline-5,6-dione (PLQ)) and enzyme (flavin adenine dinucleotide-dependent glucose dehydrogenase (FADGDH)).

66 mg of MWCNT [Ø=9.5 nm, 1.5 μm length, ≥95% purity, from Nanocyl] were dispersed in 66 ml of N,N-dimethylformamide (DMF) in a 100 ml flask and placed in a water bath under sonication for 1 h 30 min. The suspension was filtered through a PTFE filter using a vacuum pump, washed with distilled water and left for 2 hours under a hood. After filtration, the resulting buckypaper (BPE) was allowed to dry at ambient temperature against another PTFE filter for 24 hours. The buckypaper was gently detached from the filter paper and cut into individual electrodes of Ø=4 or 6 mm. The MWCNT BPE were then modified by a first addition of 20 μl of a PLQ solution (5 mm) in acetone/H₂O mixture (volume ratio 1:1) and allowed to dry for 10 minutes. 30 μl of a FADGDH enzyme solution in phosphate buffer (PB: 0.1M, pH=7.4) (10 mg·ml−1) were deposited on the surface of the modified electrode and allowed to dry for a few hours. Finally, the electrical contact was made using a metal wire fixed to the rear of the electrode with carbon paste and allowed to dry for 2 hours. The back of the electrode was insulated with silicone paste and allowed to dry for a few hours before use.
b) Preparation of the polydimethylsiloxane (PDMS) mold for the MN patch.

A master mold was produced from aluminum by micro-milling. The aluminum mold was composed of a cylindrical network of 7 mm diameter with 5×5 (25) microneedles in the shape of pencils with heights of 800, 1000 and 1200 μm respectively, a width at the base of 400 μm, and an edge-to-edge distance between the needles of 400 μm. A reverse mold was prepared by casting a mixture of PDMS and its curing agent (ratio of 10:1) on the main mold, under vacuum to remove air bubbles, and then hardened at 100° C. for 2 hours. Finally, the master and reverse molds were cooled under air for a few hours, and the PDMS mold was gently detached from the aluminum mold.
c) Preparation of the MN patch with integration of the working electrode

A PBS solution containing 20% (w/w) Dex-MA, with a DS of 9, 18, 37 or 62%, and 1% (w/w) LAP as photoinitiator was prepared in a 10 ml flask. 100 μl of solution were poured into each PDMS mold. The mold was placed in an aluminum support connected to a vacuum pump, and a reduced pressure (1-10 mbar) was applied for 2 hours to fill the mold tips. The working electrode (WE) prepared at a) was then integrated into the MN patch by inserting the electrode inside the polymer solution so that the surface of the electrode was close to the rear of the tips, and the rear of the electrode was exposed to allow the electrical connection. The MN patch with the integrated electrode WE was then allowed to dry for 24 hours. The polymer was crosslinked by a first irradiation of the back of the patch at 405 nm for 1 minute (P=75 mW/cm2), followed by demolding of the device and a last irradiation at 405 nm for 1 minute on the needle face of the patch. Finally, the electrical connection was made by following the same procedure as that described below.
d) MN patch variant with integration of a reference electrode (RE) and/or a counter-electrode (CE), and a working electrode with a CE and/or an RE (MN-RE, MN-CE, MN-RE-CE, MN-WE-RE, MN-WE-RE-CE).

An electrode RE consisting of an Ag wire chlorinated by chronoamperometry in a 1M HCl solution at 2V for 5 seconds (Ag/AgCl) or an electrode CE consisting of a Pt wire were inserted separately into a Dex-MA MN patch using the same protocol as that described at c). An electrode RE consisting of a chlorinated Ag wire and an electrode CE consisting of a Pt wire were simultaneously inserted into a Dex-MA MN patch using the same protocol as that described at c).

Three electrodes were inserted simultaneously into a Dex-MA MN patch: The BPE electrode described at b), an electrode RE consisting of a chlorinated Ag wire and an electrode CE consisting of a Pt wire. The device with 3 integrated electrodes was prepared as described at a), the 3 electrodes being inserted without contact into the viscous polymer phase before complete drying and photocrosslinking.

### Example 3

### Chronoamperometric Performance Test of a Device Comprising Several Electrodes (MN-WE-CE-RE) Integrated Into the Dex-MA MN Patch as Obtained in Example 2

Biodetection by chronoamperometric measurement was carried out at ambient temperature in a phosphate buffer (0.1 M, pH=7.4) at −0.1 V versus pseudo-reference Ag/AgCl using a Princeton Applied Research apparatus PARSTAT MC (PMC 1000/DC) running Versa Studio software with an MN patch with a degree of substitution of 37% incorporating 3 electrodes as described in example 2d (an RE electrode consisting of a chlorinated Ag wire, a self-contained platinum wire as counter-electrode (CE), and the WE electrode of example 2a)).

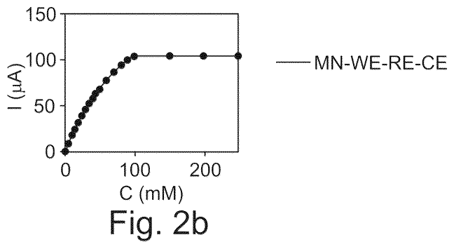

The chronoamperometry measurements are illustrated in FIG. 2a and FIG. 2b. The calibration curve in FIG. 2b is obtained from the measurements in FIG. 2a by taking the plateau values of the current obtained after each addition of glucose. A linear range of change of the current is observed with the glucose concentration over physiologically relevant concentrations. The analytical parameters are presented in Table 3.

TABLE 3

| Working | Linear | Sensitivity | Detection | |
|---|---|---|---|---|
| potential | range | (μA. mm⁻¹ · | limit | Stabilization |
| (V) | (mm); R² | cm⁻²) | LOD (μm) | time (s) |
| −0.1 | 0.3-50; 0.995 | 1.51 ± 0.03 | 300 | 83.2 ± 7.4 |

All these results demonstrate the functionality of the integrated sensor.

### Example 4

### Biocompatibility Test

NIH-3T3 murine fibroblast cells were purchased from the American Type Culture Collection (ATCC). The cells were cultured under a humidified atmosphere (90%) of 95% air/5% CO2 at 37° C., in culture medium with a high glucose content (Eagle medium supplemented with 10% fetal bovine serum and 1% antibiotics (penicillin and streptomycin) marketed by Dubelco (Gibco). The culture medium was changed every two days.

Cytotoxicity tests were performed according to ISO-10993-5 on:
1) samples of Dex-MA photocrosslinked at 405 nm for 1 minute (300 mW/cm2) (obtained by air drying at ambient temperature of aqueous solutions of Dex-MA (DS=37%) and LAP with concentrations of 20 and 1% w/w, respectively), (biocompatible hydrogel material crosslinked and swelling alone): MN sample
2) the electrodes alone, that is to say not integrated in a device according to the invention:
   the working electrode described in example 2 (WE)
   a reference electrode (RE) consisting of a chlorinated Ag wire
   a counter-electrode (CE) made of a platinum wire
3) devices according to the invention prepared according to the protocol detailed above, with one or more integrated electrodes according to the following combinations
   devices incorporating a single electrode:
      MN−WE
      MN−RE
      MN-CE
   device incorporating 2 electrodes (RE+CE): MN−RE−CE
   device incorporating 3 electrodes (WE+RE+CE): MN−WE−RE−CE

All the materials to be tested were sterilized by placing them in a 24-well plate and then immersing them entirely in an ethanol (70%)/water (30%) mixture for 3 hours. The ethanol/water solution was then withdrawn and replaced with sterile PBS for 1 hour in order to remove any trace of ethanol. The sterile PBS was then replaced with fresh sterile PBS and left for 1 hour, then sterile PBS incubated overnight (about 16 hours).

The materials were then placed in a new 24-well plate and immersed in 1 ml of culture medium ([C]=3 cm² of material per 1 ml of cell culture medium) for 24 hours, 48 hours and 7 days. Material-free control plates with cell culture medium were also provided as negative cytotoxicity controls. The culture media (release media) were then collected in their entirety, after 24 hours, 48 hours and 7 days, in Eppendorf tubes and then frozen in a freezer at −20° C.

The cells were seeded with a cell density of 5000 cells per well in a 96-well plate for 24 hours at 37.5° C. and 5% CO2. After 24 hours, the growth medium was replaced by 100 μl of release medium (thawed at 37.5° C.) or of control medium (negative control) or of cell culture medium containing 10 mm H2O2 (positive cytotoxicity control). After 24 hours, the cell culture media were removed and replaced with 100 μl of fresh cell culture medium, and 10 μl of cell proliferation reagent (WST-1 from Roche Diagnostics GmbH) were added. After 2 hours of incubation at 37.5° C. and 5% CO2, cell viability was calculated by reading off the absorbance at 450 nm, corrected for absorbance at 650 nm, with an Infinite-M1000 TECAN microplate reader according to the following formula:

$$Viability ( \% ) = \frac{{Absorbance}_{sample} - {Absorbance}_{positive control}}{{Absorbance}_{negative contol} - {Absorbance}_{positive contol}} * 100$$

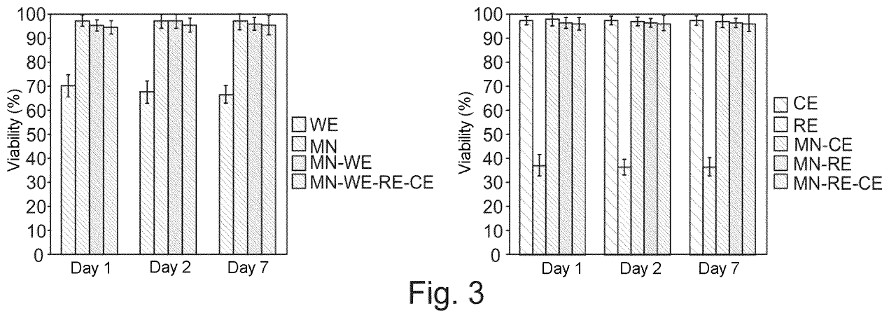

The experiments were carried out on 5 different samples and on different batches of cells and at different times. The results are presented in FIG. 3.

Cytotoxicity tests on the samples show good cytocompatibility of the microneedles at 20% w/w Dex-MA, of DS=37%, crosslinked with 1% w/w LAP (MN), with cell viability greater than 80% (94.8% to 96%), and good biocompatibility of all the devices according to the principle of the invention.

By contrast, the electrodes not integrated into the MN patch and the device exhibit cytotoxicity, with viabilities of between 67.2 and 71.8% for the WE, and between 36.1 and 36.9% for the RE.

### Example 5

### Test of the Stability of a Working Electrode Bonded to the Back of an MN Patch

A PBS solution containing 20% (w/w) Dex-MA, with a DS of 37%, and 1% (w/w) LAP as photoinitiator was prepared in a 10 ml flask. 100 μl of solution were poured into the PDMS mold. The mold was placed in an aluminum support connected to a vacuum pump, and a reduced pressure (1-10 mbar) was applied for 24 hours to fill the mold tips and dry the polymer. The dry polymer was then crosslinked under UV-vis at 405 nm for 1 minute (P=75 mW/cm2). The BPE biosensor (prepared at 2a)) was then integrated by depositing a layer of polymer solution containing 20% (w/w) of Dex-MA, with a DS of 37%, and 1% (w/w) of LAP on the biosensor and gluing the electrode to the back of the patch. The MN patch was then exposed again to UV-vis at 405 nm for 1 minute before crosslinking the adhesive layer. Finally, the electrical connection was made by following the same procedure as that described below.

The device was then analyzed by chronoamperometry at −0.1V in a 3-electrode cell employing a platinum electrode as CE, a chlorinated Ag wire as RE, the biosensor as WE, and immersing the hydrogel part of the biosensor in a PB solution with stirring for 1 hour, followed by an addition of 5 mM glucose. However, in contrast to the MN patch according to the invention and tested in example 3, no catalytic current was observed after 24 hours. Partial detachment of the electrode was also observed.

1. A device, comprising:
at least one polymeric support having a surface configured for contact with skin;
at least one array of solid polymeric microneedles integral with the support and projecting outward from the surface of the support configured for contact with the skin; and
at least one electrode,
wherein the microneedles and at least the surface of contact of the support with the skin are formed of a crosslinked hydrogel, which is non-electron-conductive in the dry state and electrolyte-conductive upon contact with an aqueous fluid,
wherein the at least one electrode is arranged in contact with the hydrogel configured to swell upon contact with an aqueous fluid and is free of direct contact with the skin, and is integrated into the constituent crosslinked hydrogel of the support which is in direct contact with the rear of the microneedles and/or integrated into the constituent crosslinked hydrogel of the microneedles.

2. The device of claim 1, in which the assembly composed of support and microneedles consists of the non-conductive crosslinked hydrogel which swells upon contact with an aqueous fluid.

3. The device of claim 1, wherein the hydrogel is free of any metallic constituent and electron-conductive polymer.

4. The device of claim 1, wherein the hydrogel is obtained by crosslinking one or more synthetic polymers, one or more biopolymers, which are optionally chemically modified to be crosslinkable, or a mixture thereof.

5. The device of claim 1, wherein the biopolymer is a polyhydroxy acid.

6. The device of claim 1, wherein the biopolymer is at least one polymer chosen from the group consisting of an alginate, hyaluronic acid, carboxymethylcellulose, chitosan, dextran, and a derivative thereof.

7. The device of claim 1, wherein the hydrogel is derived from the crosslinking of at least one dextran polymer modified by units chosen from acrylate, methacrylate, alkonyl, and alkynyl.

8. The device of claim 1, wherein the microneedles have a length of from 200 to 3000 μm.

9. The device of claim 1, wherein the at least one electrode is at least one selected from the group consisting of a reference electrode, a counter-electrode, a working electrode, an ion selective electrode, a pH electrode, an electrocatalytic electrode, a bioelectrode, and a micro- or nano-structured electrode.

10. The device of claim 1, wherein the at least one electrode is an electrode or a bioelectrode based on at least one selected from the group consisting of Pt, Au, Ag, Pd, Ni, Ir, graphitic carbon, amorphous carbon, graphene, graphene oxide, diamond, boron-doped diamond, a nanotube, a doped semiconductor fiber, a metal oxide, a conductive polymer electrode, and a conductive fiber.

11. The device of claim 1, wherein at least two electrodes are present.

12. The device of claim 1, which is a microneedle patch.

13. The device of claim 1, further comprising an electrochemical detection device attached to the at least one electrode via electrical connections.

14. A method for preparing the device of claim 1 by micro-molding, the method comprising:
crosslinking at least one polymer to form the crosslinked hydrogel,
wherein the at least one electrode is integrated by being placed in contact with the polymer prior to or simultaneously with its crosslinking.

15. The method of claim 14, wherein the polymer is a liquid or semi-liquid aqueous formulation.

16. The method of claim 14, wherein the at least one electrode is arranged on the surface of the aqueous formulation comprising the non-crosslinked polymer and is subjected to mechanical pressure in order to position it within the constituent hydrogel of the microneedles and/or at depth in proximity to the base of the microneedles prior to or simultaneously with the crosslinking.

17. The method of claim 14, wherein the crosslinking is carried out photochemically.

18. The method of claim 14, wherein the biopolymer is a polyhydroxy acid.

19. A method of electrical stimulation, comprising:
sending an electrical pulse from the device of claim 1 to a skin.

20. A method of generating energy, comprising:
electro-oxidizing a fuel in an enzymatic biofuel cell, the biofuel cell comprising the device of claim 1.

21. A method for storing energy, comprising:
charging a supercapacitor or a hybrid enzymatic biofuel cell/supercapacitor device wherein the supercapacitor or hybrid enzymatic biofuel cell/supercapacitor device comprises the device of claim 1.

22. A method for measuring an analyte in an interstitial fluid, the method comprising:
contacting the interstitial fluid with the microneedles of the device of claim 1.
